SEQUENCE LISTING (22 sequences)

SEQ ID NO: 1 - protein, 5 aa, Artificial Sequence
  CDR1 sequence
SYAMS

SEQ ID NO: 2 - protein, 17 aa, Artificial Sequence
  CDR2 sequence
AIYTSGNSTSYADSVKG

SEQ ID NO: 3 - protein, 7 aa, Artificial Sequence
  CDR3 sequence
NASNFDY

SEQ ID NO: 4 - protein, 11 aa, Artificial Sequence
  CDR1 sequence
RASQSISSYLN

SEQ ID NO: 5 - protein, 7 aa, Artificial Sequence
  CDR2 sequence
SASNLQS

SEQ ID NO: 6 - protein, 9 aa, Artificial Sequence
  CDR3 sequence
QQGSDAPAT

SEQ ID NO: 7 - protein, 5 aa, Artificial Sequence
  CDR1 sequence
DYAMH

SEQ ID NO: 8 - protein, 17 aa, Artificial Sequence
  CDR2 sequence
AITWNSGHIDYADSVEG

SEQ ID NO: 9 - protein, 116 aa, Artificial Sequence
  VH region
EVQLLESGGGLVQPGGSLRLSCAASGFTFSSYAMSWVRQAPGKGLEWVSAIYTSGNSTSYADSVKGRFTISRDNSKNTLYLQMNSLRAEDTAVYYCAKNASNFDYWGQGTLVTVSS

SEQ ID NO: 10 - protein, 107 aa, Artificial Sequence
  VL region
DIQMTQSPSSLSASVGDRVTITCRASQSISSYLNWYQQKPGKAPKLLIYSASNLQSGVPSRFSGSGSGTDFTLTISSLQPEDFATYYCQQGSDAPATFGQGTKVEIK

SEQ ID NO: 11 - protein, 243 aa, Artificial Sequence
  Sincle-chain variable fragment (scFv)
EVQLLESGGGLVQPGGSLRLSCAASGFTFSSYAMSWVRQAPGKGLEWVSAIYTSGNSTSYADSVKGRFTISRDNSKNTLYLQMNSLRAEDTAVYYCAKNASNFDYWGQGTLVTVSSGGGGSGGGGSGGGGSTDIQMTQSPSSLSASVGDRVTITCRASQSISSYLNWYQQKPGKAPKLLIYSASNLQSGVPSRFSGSGSGTDFTLTISSLQPEDFATYYCQQGSDAPATFGQGTKVEIKRAAA

SEQ ID NO: 12 - protein, 12 aa, Artificial Sequence
  CDR3 sequence
VSYLSTASSLDY

SEQ ID NO: 13 - protein, 11 aa, Artificial Sequence
  CDR1 sequence
RASQGIRNYLA

SEQ ID NO: 14 - protein, 7 aa, Artificial Sequence
  CDR2 sequence
AASTLQS

SEQ ID NO: 15 - protein, 9 aa, Artificial Sequence
  CDR3 sequence
QRYNRAPYT

SEQ ID NO: 16 - protein, 107 aa, Artificial Sequence [sequence omitted]

SEQ ID NO: 17 - protein, 121 aa, Artificial Sequence [sequence omitted]

SEQ ID NO: 18 - protein, 17 aa, Artificial Sequence
  Serine-glycine linker
SSGGGGSGGGGSGGGGS

SEQ ID NO: 19 - protein, 24 aa, Artificial Sequence
  2A peptide sequence derived from Foot and Mouth Disease Virus
APVKQTLNFDLLKLAGDVESNPGP

SEQ ID NO: 20 - DNA, 3278 bp, Artificial Sequence
  Nucleic acid sequence for bispecific antibody A7/Adalimumab
atgggctggagcctgatcctcctgttcctcgtcgctgtggctacaggtaaggggctcacagtagcaggcttgaggtctggacatatatatgggtgacaatgacatccactttgcctttctctccacaggtggcgcgcatgccgaagtgcagctggtggaatctggcggcggactggtgcagcctggcagatccctgagactgtcttgtgccgcctccggcttcaccttcgacgactacgctatgcactgggtgcgacaggcccctggcaagggactggaatgggtgtccgccatcacctggaactccggccacatcgactacgccgactctgtggaaggccggttcaccatctctcgggacaacgccaagaactccctgtacctgcagatgaacagcctgcgggccgaggacaccgccgtgtactactgtgccaaggtgtcctacctgtccaccgcctcctccctggattattggggccagggcaccctcgtgaccgtgtctagcggaggcggaggatcaggcggtggtggttcaggtggcggaggctctaccgacatccagatgacccagtccccctccagcctgtctgcctctgtgggcgacagagtgaccatcacatgccgggcctcccagggcatcagaaactacctggcctggtatcagcagaagcccggcaaggcccccaagctgctgatctacgctgcctccacactgcagtccggcgtgccctccagattctccggctctggctctggcaccgactttaccctgaccatcagctccctgcagcccgaggatgtggccacctactactgccagcggtacaacagagccccctacaccttcggacagggcaccaaggtggaaatcaaggctagcgacaagacccacacctgtcccccttgccctgcccctgagctgctgggcggaccctccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgaaaagaccatctccaaggccaagggccagccccgcgagcctcaggtgtacacactgccccccagccgggaagagatgaccaagaaccaggtgtccctgacctgcctggtcaagggcttctacccctccgatatcgccgtggaatgggagtccaacggacagcccgaaaacaactacaagaccaccccccctgtgctggactccgacggctcattcttcctgacctccaagctgaccgtggacaagtcccggtggcagcagggcaacgtgttctcctgctccgtgatgcacgaggccctgcacaaccactacacccagaagtccctgtccctgagccccggcaagacctccggcaagcccatccctaaccccctgctgggcctggactccaccagagccaagagggcaccggtgaagcagaccctgaacttcgacctgctgaagctggccggcgacgtggaatccaaccctggccctatgggctggagcctgatcctcctgttcctcgtcgctgtggctacaggtaaggggctcacagtagcaggcttgaggtctggacatatatatgggtgacaatgacatccactttgcctttctctccacaggtggcgcgcatgccgaagtgcagctgctggaatctggcggcggactggtgcagcctggcggatctctgagactgtcttgtgccgcctccggcttcaccttctccagctacgccatgtcctgggtgcgacaggctcctggcaagggcctggaatgggtgtccgccatctacacctccggcaactccacctcctacgccgactccgtgaagggccggttcaccatctcccgggacaactccaagaacaccctgtacctgcagatgaactccctgcgggccgaggacaccgccgtgtactactgtgccaagaacgcctccaacttcgactactggggccagggcaccctcgtgacagtgtctagcggaggcggaggatcaggtggcggtggatctggcggagggggctctaccgatatccagatgacccagtccccctccagcctgtctgcctctgtgggcgacagagtgaccatcacctgtcgggcctcccagtccatctcctcctacctgaactggtatcagcagaagcccggcaaggcccccaagctgctgatctactccgccagcaacctgcagtccggcgtgccctccagattctccggctctggctctggcaccgactttaccctgaccatcagctccctgcagcctgaggacttcgccacctactactgccagcagggctctgatgcccctgccacctttggacagggcaccaaggtggaaatcaagcgggctgccgctgctagcgataagacacatacctgccccccctgtccagctcccgaactgctgggaggcccttctgtgtttctgtttccacccaagcctaaagatacactgatgatcagccgcacccctgaagtcacatgtgtggtggtcgatgtgtctcatgaagatcccgaagtcaagtttaactggtatgtggatggggtcgaggtccacaatgctaagacaaagcctcgggaagaacagtataactccacatatcgcgtcgtgtctgtcctgacagtcctgcatcaggattggctgaatgggaaagaatacaaatgtaaagtctctaacaaggctctgcccgctcctatcgagaaaaccatcagcaaggctaaaggacagcccagagaaccccaggtctacaccctgcctccatctcgcgaggaaatgacaaaaaatcaggtgtcactgtattgtctggtcaaagggttttaccccagcgacattgccgtcgagtgggagagcaatggccagcctgaaaacaattataagacaacacctcccgtcctggacagcgacggatcattttttctgtactctaagctgacagtcgacaagagcagatggcagcagggaaatgtctttagctgcagcgtgatgcatgaagctctgcataatcattatacccagaagagtctgagcctgtcccccggcaagggcggctctcaccaccatcatcaccac

SEQ ID NO: 21 - protein, 508 aa, Artificial Sequence
  Amino acid sequence for bispecific antibody A7/Adalimumab, Adalimumab chain
MGWSLILLFLVAVATVARMEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSTDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKTSGKPIPNPLLGLDST

SEQ ID NO: 22 - protein, 500 aa, Artificial Sequence
  Amino acid sequence for bispecific antibody A7/Adalimumab, A7 chain
MGWSLILLFLVAVATVARMEVQLLESGGGLVQPGGSLRLSCAASGFTFSSYAMSWVRQAPGKGLEWVSAIYTSGNSTSYADSVKGRFTISRDNSKNTLYLQMNSLRAEDTAVYYCAKNASNFDYWGQGTLVTVSSGGGGSGGGGSGGGGSTDIQMTQSPSSLSASVGDRVTITCRASQSISSYLNWYQQKPGKAPKLLIYSASNLQSGVPSRFSGSGSGTDFTLTISSLQPEDFATYYCQQGSDAPATFGQGTKVEIKRAAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGSHHHHHH

(19) United States
(12) Patent Application Publication
(10) Pub. No.: US 2016/0215047 A1
(43) Pub. Date: Jul. 28, 2016

(54) BISPECIFIC ANTIBODY AGAINST TNF-ALPHA AND SYNOVIAL MICROVASCULATURE OF ARTHRITIS PATIENTS

(71) Applicant: Queen Mary University of London, London (GB)

(72) Inventor: Constantino PITZALIS, London (GB)

(21) Appl. No.: 14/916,936

(22) PCT Filed: Sep. 4, 2014
(86) PCT No.: PCT/GB2014/052681

§ 371 Date: Mar. 4, 2016

(30) Foreign Application Priority Data
Sep. 5, 2013 (GB) 1315788.8

(51) Int. Cl. C07K 16/24; C07K 16/18

(52) U.S. Cl. CPC C07K 16/241; C07K 16/18; C07K 2317/565; C07K 2317/622; C07K 2317/21; A61K 2039/505

(57) ABSTRACT

The present invention provides bispecific molecule comprising: (i) a first antigen binding portion which specifically targets the synovial microvasculature of arthritis patients and which binds to the same epitope as an antigen binding polypeptide comprising the amino acid sequence shown as SEQ ID No 11; and (ii) a second antigen binding portion which binds tumour necrosis factor alpha (TNF-α). The present invention also relates to the use of such bispecific molecules in the prevention and/or treatment of arthritis.

### FIELD OF THE INVENTION

The present invention relates to a bispecific molecule which specifically targets the synovial microvasculature of arthritis patients. The molecule comprises a targeting function which targets the molecule to the synovial microvasculature and an effector function which binds tumour necrosis factor alpha (TNF-α).

### BACKGROUND TO THE INVENTION

Rheumatoid arthritis (RA) is one of the most common autoimmune diseases and a leading cause of chronic pain affecting over three million people in Europe alone. Rheumatoid arthritis affects 1 to 2% of the population. According to Medical Expenditure Panel Survey (MEPS) data, in the US the total costs incurred towards the treatment of rheumatoid arthritis and related arthritis in 2003 was $128 billion; the average per person cost is currently $8500. Each year, arthritis and its associated complications results in over 750,000 hospitalizations and 36 million outpatient visits. Up to 15% of people inflicted with any type of arthritis suffer from a reduction in the amount of physical activities they can perform. Typically when physical activity is reduced patients tend to develop depression because of their lack of independence and freedom.

In the UK there are around 400,000 adults with rheumatoid arthritis and arthritis is the most common condition for which people receive Disability Living Allowance. Over half a million people receive DLA as a result of arthritis (representing more than 18 percent of all DLA claimants), which is more than the total for heart disease, stroke, chest disease and cancer combined.

RA is an inflammatory disease of the synovial joints, which generally affects wrists, fingers, knees, feet, and ankles on both sides of the body. RA causes inflammation of the synovial membranes that line and protect the joints and tendons and, allow smooth and free movement of joints. Inflammation of the synovial membranes causes swelling of the affected joints and eventually leads to progressive cartilage destruction and erosion of bone, impairing range of movement and leading to deformity.

RA is an on-going, progressive disease that also affects other organs of the body and can result in profound disability and life threatening complications. Hence, RA is a major cause of disability with a significant associated morbidity and mortality.

The onset age of RA is variable, ranging from children to individuals in their 90s. The prevalence of RA in populations of Western Europe and USA is approximately 1% with a female to male ratio of 3:1. Further, the total annual economic impact of rheumatoid arthritis is estimated at approximately £35 billion in Western Europe.

Therapy for RA has been significantly improved in the last decade by the introduction of recombinant antibodies targeting a range of cytokines, T cells and B cells.

Adalimumab is a recombinant fully human IgG1 monoclonal antibody which binds to TNF-α with high specificity. It is indistinguishable structurally and functionally from naturally occurring human IgG1 making it suitable for long-term administration with low immunogenicity. It is composed of heavy- and light-chain variable regions and IgG1:κ constant regions engineered through phage display technology. Adalimumab binds to a single epitope on the N-terminus of TNF-α and blocks its interaction with the p55 and p75 cell surface TNF receptors.

Adalimumab is prescribed for a number of inflammatory diseases including rheumatoid arthritis, psoriatic arthritis, juvenile idiopathic arthritis, psoriasis, ankylosing spondylitis, Crohn's disease and ulcerative colitis. The recommended dose for adult patients with rheumatoid arthritis is 40 mg administered fortnightly as a subcutaneous injection. The estimated annual cost for this regimen is over £9,000.

Because TNF-α normally plays an important role in protecting the body from infections, treatment with TNF inhibitors can have serious side effects. Patients treated with adalimumab are at increased risk for developing infections from opportunistic bacterial, fungal, viral and parasitic pathogens. Activation of previously undetected tuberculosis infections and reactivation of hepatitis B virus have been reported. Due to these risks, adalimumab is generally not prescribed to patients with active infections. TNF inhibitors have also been reported to exacerbate multiple sclerosis, congestive heart failure and certain autoimmune conditions such as lupus. In young patients, treatment with adalimumab or similar medications has been associated with life-threatening lymphomas such as hepatosplenic T cell lymphoma.

Therefore, there is still a major unmet clinical need in RA and a requirement for alternative therapeutic options having a greater frequency of remission induction and improved safety profile with less systemic toxicity.

### DESCRIPTION OF THE FIGURES

FIG. 1—Cloning strategy for bispecific A7/Adalimumab antibody A. Cloning strategy for A7 scFv-Fc and Adalimumab scFv-Fc cloning in a single monocystronic sequence. B. Schematic of bispecific A7/Adalimumab construct

FIG. 2—Analysis of heterodimerisation A. scFv-Fc-Fc schematic of antibody construct bearing a single scFv Adalimumab domain. B. Possible dimerisation outcome of asymmetric antibody construct.

FIG. 3—IHC staining on formalin fixed human arthritic synovium Reactivity of A7 scFv-Fc, Adalimumab scFv-Fc and bispecific A7/Adalimumab with sections of human arthritic synovium was examined using biotinylated antibodies and detected with streptavidin-HRP.

FIG. 4—Immuno-fluorescent staining on frozen human arthritic synovium Reactivity of A7 scFv-Fc, Adalimumab scFv-Fc and bispecific A7/Adalimumab with sections of human arthritic synovium was examined using biotinylated antibodies and detected with streptavidin-ALEXA fluor 488 (green) in the presence of an anti-vWF antibody (red). A7 reactivity is confined in the vascular region of the synovium (green). The bispecific antibody A7/Adalimumab shows a similar reactivity on the synovium of A7 scFv-Fc, demonstrating the functional activity of the A7 portion.

FIG. 5—Nucleic acid sequence for bispecific antibody A7/Adalimumab (optimised for CHO expression)

FIG. 6—Amino acid sequence for bispecific antibody A7/Adalimumab

### SUMMARY OF ASPECTS OF THE INVENTION

The present inventors have produced a bispecific antibody which comprises a targeting portion which specifically targets the synovial microvasculature of arthritis patients and an effector portion which has the same binding specificity as Adalimumab.

In a first aspect the present invention provides a bispecific molecule comprising:
(i) a first antigen binding portion which specifically targets the synovial microvasculature of arthritis patients and which binds to the same epitope as an antigen binding polypeptide comprising the amino acid sequence shown as SEQ ID No 11; and
(ii) a second antigen binding portion which binds tumour necrosis factor alpha (TNF-α).

The first antigen binding portion may comprise:
a) a heavy chain variable region comprising
   (i) a CDR1 comprising the sequence SYAMS (SEQ ID No. 1);
   (ii) a CDR2 comprising the sequence AIYTSGNSTSYADSVKG (SEQ ID No 2); and
   (iii) a CDR3 comprising the sequence NASNFDY (SEQ ID No 3), and
b) a light chain variable region comprising
   (i) a CDR1 comprising the sequence RASQSISSYLN (SEQ ID No. 4);
   (ii) a CDR2 comprising the sequence SASNLQS (SEQ ID No. 5); and
   (iii) a CDR3 comprising QQGSDAPAT (SEQ ID No. 6)
      or a variant of any one or more of those CDR sequences having one, two or three amino acid variations from the given sequence, provided that the first antigen binding portion retains the ability to bind to the same epitope as an antigen binding polypeptide comprising the amino acid sequence shown as SEQ ID No 11.

The first antigen binding portion may comprise a VL sequence as shown in SEQ ID No. 9 and a VH sequence as shown in SEQ ID No. 10, or a variant thereof having at least 80% sequence identity which specifically targets the synovial microvasculature of arthritis patients and which binds to the same epitope as an antigen binding polypeptide comprising the amino acid sequence shown as SEQ ID No 11.

The second antigen binding portion may comprise:
a) a heavy chain variable region comprising
   (i) a CDR1 comprising the sequence DYAMH (SEQ ID No. 7);
   (ii) a CDR2 comprising the sequence AITWNSGHIDYADSVEG (SEQ ID No 8); and
   (iii) a CDR3 comprising the sequence VSYLSTASSLDY (SEQ ID No 12), and
b) a light chain variable region comprising
   (i) a CDR1 comprising the sequence RASQGIRNYLA (SEQ ID No. 13);
   (ii) a CDR2 comprising the sequence AASTLQS (SEQ ID No. 14); and
   (iii) a CDR3 comprising the sequence QRYNRAPYT (SEQ ID No. 15)
      or a variant of any one or more of those CDR sequences having up to three amino acid variations from the given sequence, provided that the second antigen binding portion retains the ability to bind TNF-α.

The second antigen binding portion may comprise a VL sequence as shown in SEQ ID No. 16 and a VH sequence as shown in SEQ ID No. 17 or a variant thereof having at least 80% sequence identity which is capable of binding TNFα.

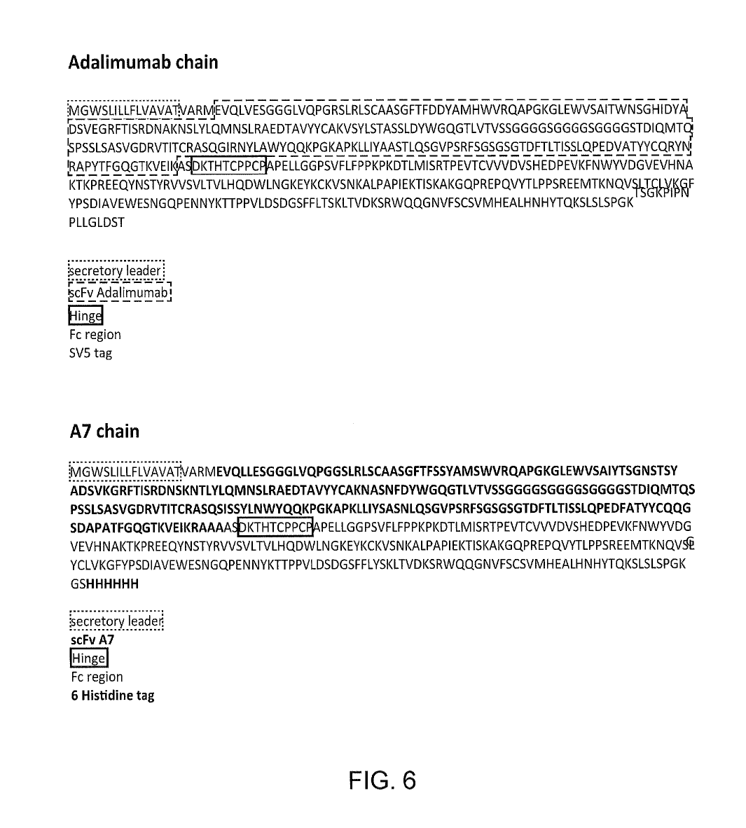

The bispecific molecule may comprise an amino acid sequence having at least 80% identity to the amino acid sequence shown in FIG. 6 (Adalimumab chain and/or A7 chain). The amino acid sequence may have at least 85%, 90%, 95%, 98% or 99% identity to the Adalimumab chain and/or the A7 chain amino acid sequence shown in FIG. 6.

The bispecific molecule may be an scFv.

The bispecific molecule may be a bispecific human antibody.

In a second aspect, the present invention provides a bispecific molecule according to the first aspect of the invention for use in the treatment of arthritis.

In a third aspect, the present invention provides a method for treating arthritis in a subject, which comprises the step of administering a bispecific molecule according to the first aspect of the invention to a subject.

The method may be used for treating, for example, osteoarthritis and/or rheumatoid arthritis.

In a fourth aspect the present invention provides a method for producing a bispecific molecule according to the first aspect of the invention, which method comprises the step of conjugating the first antigen binding portion to the second antigen binding portion.

In a fifth aspect, the present invention provides a nucleic acid sequence encoding a bispecific molecule according to the first aspect of the invention.

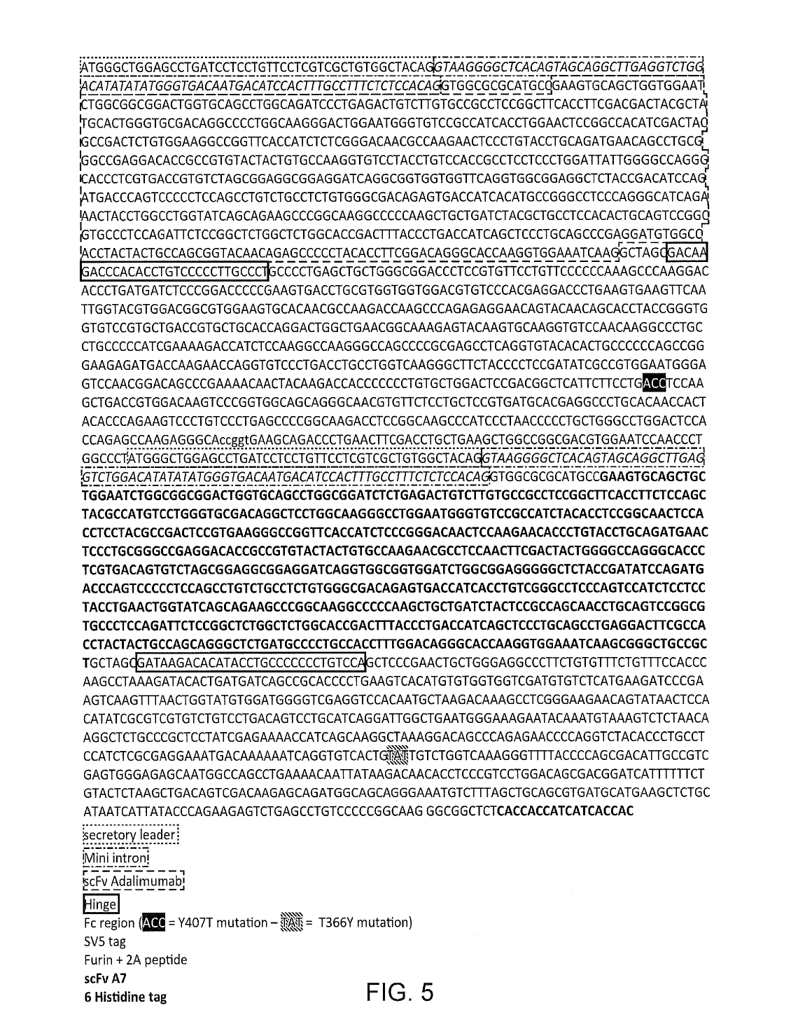

The nucleic acid sequence of the invention may have at least 80% identity to the nucleic acid sequence shown in FIG. 5. The nucleic acid sequence may have at least 85%, 90%, 95%, 98% or 99% identity to the nucleic acid sequence shown in FIG. 5.

In a sixth aspect, the present invention provides a vector comprising a nucleic acid sequence according to the fifth aspect of the invention.

In a seventh aspect, the present invention provides a host cell comprising a vector according to the sixth aspect of the invention.

The bispecific molecule of the present invention addresses many of the problems associated with the use of Adalimumab for the treatment of arthritis. For example, since the targeting portion specifically targets the synovial microvasculature of arthritis patients, it is possible to use a lower effective concentration of Adalimumab for treatment, relating to cost savings. Also, the targeting effect means that non-specific TNF inhibition is minimised, reducing the risk of side effects such as opportunistic infections, heart conditions and autoimmune disease.

### DETAILED DESCRIPTION

### Bispecific Molecule

A multispecific antibody is an antibody that can bind to at least two different antigen epitopes. The molecule of the present invention is “bispecific” in the sense that it binds at least two different antigen epitopes, namely:

(i) the epitope recognised by an antibody comprising the amino acid sequence shown as SEQ ID No 11; and

(ii) an epitope on tumour necrosis factor alpha (TNF-α).

The molecule of the present invention may have additional binding specificities, making it tri- or multi-specific.

Methods for making bispecific antigen-binding polypeptides are known in the art. Early approaches to bispecific antibody engineering included chemical crosslinking of two different antibodies or antibody fragments and quadromas.

Quadromas resemble monoclonal antibodies with two different antigen binding arms. They are generated by fusing two different hybridoma cells each producing a different monoclonal antibody. The antibody with the desired bispecificity is created by random pairing of the heavy and light chain.

TriomAbs are bispecific, trifunctional antibodies with each arm binding to a different antigen epitope and the Fc domain binding to FcR-expressing cells such as NK cells or dendritic cells. They are produced by a quadroma cell line prepared by the fusion of two specific hybridoma cell lines which allows the correct association of the heavy and light chain of each specificity without production of inactive heteromolecules.

ScFv fragments can be made bispecific using a number of approaches. ScFv molecules can be engineered in the VH-VL or VL-VH orientation with a linker varying in size to ensure that the resulting scFv forms stable monomers or multimers. When the linker size is sufficiently small for example 3 to 12 residues, the scFv cannot fold into a functional monomer. Instead, it associates with another scFv to form a bivalent dimer. When the linker size is further reduced, trimers and tetramers can form.

Diabodies are dimeric scFvs where the VH and VL domains of two antibodies A and B are fused to create the two chains VHA-VLB and VHB-VLA linked together by a peptide linker. The antigen binding sites of both antibodies A and B are recreated giving the molecules its bispecificity. Single-chain diabodies (sc-diabodies) have an additional linker connecting the VHA-VLB and VHB-VLA fragments. Tandem scFv consists of two sc-diabodies connected by a flexible peptide linker on a single protein chain. Another bispecific scFv format, the bispecific T-cell engager (BiTE) consists of two scFv fragments joined via a flexible linker where one fragment is directed against a surface antigen and the other against CD3 on T cells. Miniantibodies are generated by the association of two scFv fragments through modified dimerisation domains using a leucine zipper.

The scFv-Fc antibody is an IgG-like antibody with human IgG1 hinge and Fc regions (CH2 and CH3 domains). Each scFv arm can have a different specificity making the molecule bispecific. One method of generating an scFv-Fc heterodimer is by adopting the Knobs-into-Holes technology. Knobs are created by replacing small amino side chains at the interface between CH3 domains with larger ones, whereas holes are constructed by replacing large side chains with smaller ones.

### Antigen Binding Portion

The bispecific molecule of the first aspect of the invention comprises at least two antigen binding portions.

The term “antigen-binding portion” is used to mean a polypeptide which comprises one or more complementarity determining regions (CDRs) and binds antigen in the same way as antibody or antibody-like molecule.

A classical antibody molecule comprises four polypeptide chains: two heavy (H) chains; and two light (L) chains inter-connected by disulfide bonds. Each heavy chain is comprised of a heavy chain variable region (VH) and a heavy chain constant region. The heavy chain constant region is comprised of three domains, CH1, CH2 and CH3. Each light chain is comprised of a light chain variable region (VL) and a light chain constant region. The light chain constant region is comprised of one domain, CL. The VH and VL regions can be further subdivided into regions of hypervariability, termed complementarity determining regions (CDRs) interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In a classical antibody molecule, the pairing of heavy and light chains brings together the CDRs from each chain to create a single hypervariable surface which forms the antigen-binding site at the tip of each of the Fab arms. It is common for only a subset of the six total CDRs to contribute to antigen binding. For example when the antibody MOPC 603 binds to phosphochlorine the light-chain variable region contributes only CDR3 to the binding site, whereas all three CDRs from the heavy chain are involved.

It is also possible for a single VH or VL chain to bind antigen, for example in domain antibodies (dAbs—see below).

The term “antibody” includes intact antibodies, fragments of antibodies, e.g., Fab, F(ab′) 2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced IL 13 binding and/or reduced FcR binding).

The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Binding fragments include Fab, Fab′, F(ab′) 2, Fabc, Fd, dAb, Fv, single chains, single-chain antibodies, e.g., scFv, single domain antibodies, an isolated complementarity determining region (CDR), a UniBody, a domain antibody and a Nanobody.

A Fab fragment is a monovalent fragment consisting of the VL, VH, CL and CH1 domains. A F(ab′)2 fragment is a bivalent fragment comprising two Fab fragments linked by a disulfide bridge at the hinge region. An Fd fragment consists of the VH and CH 1 domains, and an Fv fragment consists of the VL and VH domains of a single arm of an antibody.

A dAb fragment consists of a single VH domain or VL domain which alone is capable of binding an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

The antigen-binding portion may be based on an scFv fragment. In a classical antibody molecule, the two domains of the Fv fragment, VL and VH, are coded for by separate genes. However they can be joined, using recombinant methods, by a synthetic linker that enables them to be made as a single protein chain known as single chain Fv (scFv) in which the VL and VH regions pair to form monovalent molecules.

Antibody-like molecules include the use of CDRs separately or in combination in synthetic molecules such as SMIPs and small antibody mimetics. Specificity determining regions (SDRs) are residues within CDRs that directly interact with antigen. The SDRs correspond to hypervariable residues. CDRs can also be utilized in small antibody mimetics, which comprise two CDR regions and a framework region.

An antibody or binding portion thereof also may be part of a larger immunoadhesion molecules formed by covalent or non-covalent association of the antibody or antibody portion with one or more other proteins or peptides. Examples of such immunoadhesion molecules include use of the streptavidin core region to make a tetrameric scFv molecule and use of a cysteine residue, a marker peptide and a C-terminal polyhistidine tag to make bivalent and biotinylated scFv molecules.

The antigen-binding portion may be based on an antibody mimetic, such as: an Affibody, a DARPin, an Anticalin, an Avimer, a Versabody and a Duocalin.

### CDRs

The antigen-binding portions of the present invention may comprise complementarity determining region(s) (CDR(s)).

The first antigen binding portion may comprise

(i) a heavy chain CDR1:SYAMS (SEQ ID No. 1);

(ii) a heavy chain CDR2: AIYTSGNSTSYADSVKG (SEQ ID No 2);

(iii) a heavy chain CDR3:NASNFDY (SEQ ID No 3);

(iv) a light chain CDR1: RASQSISSYLN (SEQ ID No. 4);

(ii) a light chain CDR2: SASNLQS (SEQ ID No. 5); and

(iii) a light chain CDR3: QQGSDAPAT (SEQ ID No. 6).

The first antigen binding portion may comprise a variant of one, two, three, four, five or all six of those CDR sequences having one, two or three amino acid variations from the given sequence, provided that the first antigen binding portion retains the ability to bind to the same epitope as an antigen binding polypeptide comprising the amino acid sequence shown as SEQ ID No 11.

The second antigen binding portion may comprise

(i) a heavy chain CDR1: DYAMH (SEQ ID No. 7);

(ii) a heavy chain CDR2: AITWNSGHIDYADSVEG (SEQ ID No 8);

(iii) a heavy chain CDR3: VSYLSTASSLDY (SEQ ID No 12);

(iv) a light chain CDR1: RASQGIRNYLA (SEQ ID No. 13);

(ii) a light chain CDR2: AASTLQS (SEQ ID No. 14); and

(iii) a light chain CDR3: QRYNRAPYT (SEQ ID No. 15).

The second antigen binding portion may comprise a variant of one, two, three, four, five or all six of those CDR sequences having one, two or three amino acid variations from the given sequence, provided that the second antigen binding portion retains the ability to bind TNF-α.

### V Regions

The first antigen binding portion may comprise a VH region as shown in SEQ ID No. 9 or a variant thereof having, for example, at least 70, 80, 90, 95 or 99% sequence identity which, optionally in combination with a light chain, specifically targets the synovial microvasculature of arthritis patients and which binds to the same epitope as an antigen binding polypeptide comprising the amino acid sequence shown as SEQ ID No 11.

### SEQ ID No 9:

EVQLLESGGGLVQPGGSLRLSCAASGFTFSSYAMSWVAQAPGKGLEWVSA
IYTSGNSTSYADSVKGRFTISRDNSKNTLYLQMNSLRAEDTAVYYCAKNA
SNFDYWGQGTLVTVSS

The first antigen binding portion may comprise a VL region as shown in SEQ ID No. 10 or a variant thereof having, for example, at least 70, 80, 90, 95 or 99% sequence identity which, optionally in combination with a heavy chain, specifically targets the synovial microvasculature of arthritis patients and which binds to the same epitope as an antigen binding polypeptide comprising the amino acid sequence shown as SEQ ID No 11.

### SEQ ID No 10:

DIQMTQSPSSLSASVGDRVTITCRASQSISSYLNWYQQKPGKAPKLLIYS
ASNLQSGVPSRFSGSGSGTDFTLTISSLQPEDFATYYCQQGSDAPATFGQ
GTKVEIK

For both the VH and VL regions, variations in the sequence may be concentrated in the framework regions of the polypeptide. The CDRs may comprise relatively few amino acid substitutions.

The second antigen binding portion may comprise a VL region as shown in SEQ ID No. 16 or a variant thereof having, for example, at least 70, 80, 90, 95 or 99% sequence identity which, optionally in combination with a heavy chain, is capable of binding TNFα.

### SEQ ID No. 16:

DIQMTQSPSSLSASVGDRVTITCRASQGLIZNYLAWYQQKPGKAPKLLIY
AASTLQSGVPSRFSGSGSGTDFTLTISSLQPEDVATYYCQRYNRAPYTFG
QGTKVEIK

The second antigen binding portion may comprise a VH sequence as shown in SEQ ID No. 17 or a variant thereof having, for example, at least 70, 80, 90, 95 or 99% sequence identity which, optionally in combination with a heavy chain, is capable of binding TNFα.

### SEQ ID No. 17:

EVQLVESGGGLVQPGRSLRLSCAASGFTEDDYAMHWVRQAPGKGLEWVSA
ITWNSGHIDYADSVEGRFTISRDNAKNSLYLQMNSLRAEDTAVYYCAKVS
YLSTASSLDYWGQGTLVTVSS

The first antigen binding portion may be an scFv having the sequence shown as SEQ ID No 11 or a variant thereof having, for example, at least 70, 80, 90, 95 or 99% sequence identity which specifically targets the synovial microvasculature of arthritis patients and which binds to the same epitope as an antigen binding polypeptide comprising the amino acid sequence shown as SEQ ID No 11.

### SEQ ID No 11:

EVQLLESGGGLVQPGGSLRLSCAASGFTFSSYAMSWVRQAPGKGLEWVSA
IYTSGNSTSYADSVKGRFTISRDNSKNTLYLQMNSLRAEDTAVYYCAKNA
SNFDYWGQGTLVTVSSGGGGSGGGGSGGGGSTDIQMTQSPSSLSASVGDR
VTITCRASQSISSYLNWYQQKPGKAPKLLIYSASNLQSGVPSRFSGSGSG
TDFTLTISSLQPEDFATYYCQQGSDAPATFGQGTKVEIKRAAA

Again, variations in the sequence may be concentrated in the framework regions and linker region of the polypeptide. The CDRs may comprise relatively few amino acid substitutions.

### Sequence Comparisons

Identity comparisons can be conducted by eye, or more usually, with the aid of readily available sequence comparison programs. These commercially available computer programs can calculate % identity between two or more sequences. A suitable computer program for carrying out such an alignment is the GCG Wisconsin Bestfit package. Examples of other software than can perform sequence comparisons include, but are not limited to, the BLAST package, FASTA and the GENEWORKS suite of comparison tools. Both BLAST and FASTA are available for offline and online searching.

The sequence may have one or more deletions, insertions or substitutions of amino acid residues which produce a silent change and result in a functionally equivalent molecule. Deliberate amino acid substitutions may be made on the basis of similarity in polarity, charge, solubility, hydrophobicity, hydrophilicity, and/or the amphipathic nature of the residues as long as the activity is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

### Human Antibody

The antigen binding portions may be non-human, chimaeric, humanised or fully human.

Non-human antibodies include polyclonal or monoclonal antibody preparations from mouse, rat, rabbit, sheep, goat or other mammals.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. A crude polyclonal antibody preparation may be obtained by immunising an animal with antigen.

Chimeric antibodies comprise sequences from at least two different species. As one example, recombinant cloning techniques may be used to include variable regions, which contain the antigen-binding sites, from a non-human antibody (i.e., an antibody prepared in a non-human species immunized with the antigen) and constant regions derived from a human immunoglobulin.

The antigen binding portions may be humanized.

“Humanized” forms of non-human (e.g., murine) antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also may comprise at least a portion of an immunoglobulin constant region (Fe), typically that of a human immunoglobulin.

The antigen binding portions may be fully human, as is the case for the scFv described in the Examples.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No, 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations may be introduced, for example, using a selective mutagenesis approach. A human antibody may have at least one position replaced with an amino acid residue, e.g., an activity enhancing amino acid residue, which is not encoded by the human germline immunoglobulin sequence. A human antibody may have some amino acid changes within the CDR regions. However, the term “human antibody” as used herein is not intended to include antibodies in which CDR sequences derived from the germline of another mammalian species, such as a mouse, have been grafted onto human framework sequences.

Fully human recombinant antibodies are likely to be considerably less immunogenic than non-human (e.g. murine), chimeric or humanised antibodies when used for therapy as they comprise effectively no foreign sequence.

### Reactivity

The bispecific molecule of the present invention specifically targets the microvasculature of arthritis patients. For example, the antigen binding polypeptide may target the microvasculature of osteoarthritis or rheumatoid arthritis (RA) patients.

In a normal joint, the synovial membrane lines the non-weight bearing aspects of the joint. In arthritis, the synovium becomes infiltrated by T-helper cells, B cells, macrophages and plasma cells. Extensive angiogenesis occurs in the synovium, significantly increasing the microvasculature. The antigen binding polypeptide of the present invention exhibits specific reactivity with this synovial microvasculature.

The bispecific molecule may react with the stromal (i.e. connective tissue) compartment of the microvasculature. The stromal compartment of the microvasculature is attractive for antibody-based targeting applications, since the compartment is stable and present in abundance.

The bispecific molecule may react with pericytes. Pericytes, also known as Rouget cells or mural cells, are associated abluminally with all vascular capillaries and post-capillary venules. Pericyte specificity may be investigated by dual staining with a pericyte-specific marker such as NG2.

The bispecific molecule may bind the cell surface of the smooth muscle cells found in the synovial microvasculature.

The bispecific molecule may exhibit perivascular reactivity, i.e. it may preferentially bind to sites around the blood vessels within the synovial microvasculature.

The bispecific molecule of the present invention “specifically targets” the synovial vasculature of arthritis patients in the sense that, following administration to a patient, the bispecific molecule exhibits a preferential binding capacity to synovium as opposed to other tissue (e.g. skin). The bispecific molecule may exhibit a two-three- or four-fold preferential binding capacity for arthritic synovium to other tissues.

The bispecific molecule of the present invention should not exhibit significant reactivity with vital organs, such as heart, liver, lung, pancreas, cerebral cortex and digestive system.

The bispecific molecule of the present invention should not exhibit significant reactivity with normal tissue such as lymph, thymus, adrenal gland, ovary and testis.

The bispecific molecule of the present invention should not significantly target normal, non-arthritic joints. For example, when administered to an arthritis patient who has a combination of arthritic and normal joints, the bispecific molecule should preferentially target to the arthritic joints. The bispecific molecule may preferentially target and/or accumulate at joints showing the highest amount of synovial angiogenesis.

Reactivity and/or targeting is considered “significant” if it renders a therapeutic product based on the antigen-binding polypeptide unsafe or ineffective for use due to low levels of specificity.

The bispecific molecule of the present invention also binds TNFα through the second antigen binding portion.

Tumor necrosis factor-α (TNF-α) is a cytokine central to many aspects of the inflammatory response. Macrophages, mast cells, and activated T_{H} cells (especially T_{H}1 cells) secrete TNF-α. TNF-α stimulates macrophages to produce cytotoxic metabolites, thereby increasing phagocytic killing activity.

TNF-α has been implicated in numerous autoimmune diseases. Rheumatoid arthritis, psoriasis, and Crohn's disease are three disorders in which inhibition of TNF-α has demonstrated therapeutic efficacy. Rheumatoid arthritis illustrates the central role of TNF-α in the pathophysiology of autoimmune diseases. Macrophages in a diseased joint secrete TNF-α, which activates endothelial cells, other monocytes, and synovial fibroblasts. Activated endothelial cells up-regulate adhesion molecule expression, resulting in recruitment of inflammatory cells to the joint. Monocyte activation has a positive feedback effect on T-cell and synovial fibroblast activation. Activated synovial fibroblasts secrete interleukins, which recruit additional inflammatory cells. With time, the synovium hypertrophies forms a pannus that leads to destruction of bone and cartilage in the joint, causing the characteristic deformity and pain of rheumatoid arthritis.

Binding of the bispecific molecule to TNFα though the second binding portion may prevent or inhibit the activation of TNF receptors. The bispecific molecule may bind to an epitope on the N-terminus of TNFα. The bispecific molecule may block the interaction of TNFα with the p55 and p75 cell surface TNF receptors.

### Nucleic Acid Sequence

The present invention also provides a nucleotide sequence capable of encoding a bispecific molecule according to the present invention.

The nucleotide sequence may be natural, synthetic or recombinant. It may be double or single stranded, it may be DNA or RNA or combinations thereof. It may, for example, be cDNA, PCR product, genomic sequence or mRNA.

The nucleotide sequence may be codon optimised for production in the host/host cell of choice.

It may be isolated, or as part of a plasmid, vector or host cell.

The percent identity between two nucleotide sequences can be determined by comparing a position in each sequence that may be aligned for purposes of comparison. Expression as a percentage of identity refers to a function of the number of identical nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g. default settings. ENTREZ is available through the National Center for Biotechnology Information, National Library of Medicine, National Institutes of Health, Bethesda, Md. The percent identity of two sequences may be determined by the GCG program with a gap weight of 1, e.g. each gap is weighted as if it were a single nucleotide mismatch between the two sequences.

The variant sequence may comprise one or more nucleotide substitutions, insertions or deletions. Nucleotide substitutions may be “silent” such that the codon encodes the same amino acid due to the degeneracy in the genetic code.

Where nucleotide substitutions cause a change in the encoded amino acid sequence, these may be concentrated in the framework regions and linker region of the polypeptide. The regions encoding the CDRs may comprise relatively few mutations.

### Vector

The term “vector” refers to a nucleic acid molecule capable of transporting another nucleic acid to which it has been linked. One type of vector is an episome, i.e., a nucleic acid capable of extra-chromosomal replication. Another type of vector is an integrative vector that is designed to recombine with the genetic material of a host cell. Vectors may be both autonomously replicating and integrative, and the properties of a vector may differ depending on the cellular context (i.e., a vector may be autonomously replicating in one host cell type and purely integrative in another host cell type). Vectors capable of directing the expression of expressible nucleic acids to which they are operatively linked are referred to as “expression vectors.”

A plasmid is an extra-chromosomal DNA molecule separate from the chromosomal DNA which is capable of replicating independently of the chromosomal DNA. They are usually circular and double-stranded.

Plasmids may be used to express a protein in a host cell. For example a bacterial host cell may be transfected with a plasmid capable of encoding a particular protein, in order to express that protein. The term also includes yeast artificial chromosomes and bacterial artificial chromosomes which are capable of accommodating longer portions of DNA.

### Host Cell

The present invention further provides cells and cell lines capable of producing bispecific molecule of the invention. Representative host cells include bacterial, yeast, mammalian and human cells, such as CHO cells, HEK-293 cells, HeLa cells, CV-1 cells, and COS cells. Methods for generating a stable cell line following transformation of a heterologous construct into a host cell are known in the art. Representative non-mammalian host cells include insect cells. Antibodies may also be produced in transgenic animals.

### Therapeutic Method

The bispecific molecule of the present invention may be used in the treatment of arthritis or rheumatic diseases.

Arthritis is a general term relating to diseases characterised by cute or chronic inflammation of one or more joints, usually accompanied by pain and stiffness, resulting from infection, trauma, degenerative changes, autoimmune disease, or other causes.

Osteoarthritis, also known as degenerative arthritis or degenerative joint disease, is a group of mechanical abnormalities involving degradation of joints, including articular cartilage and subchondral bone. Symptoms may include joint pain, tenderness, stiffness, locking, and sometimes an effusion. A variety of causes—hereditary, developmental, metabolic, and mechanical—may initiate processes leading to loss of cartilage.

Rheumatoid arthritis (RA) is a chronic, systemic inflammatory disorder that may affect many tissues and organs, but principally attacks synovial joints. The process produces an inflammatory response of the synovium (synovitis) secondary to hyperplasia of synovial cells, excess synovial fluid, and the development of pannus in the synovium. The pathology of the disease process often leads to the destruction of articular cartilage and ankylosis of the joints. Rheumatoid arthritis can also produce diffuse inflammation in the lungs, pericardium, pleura, and sclera, and also nodular lesions, most common in subcutaneous tissue under the skin. Although the cause of rheumatoid arthritis is unknown, autoimmunity plays a pivotal role in both its chronicity and progression, and RA is considered as a systemic autoimmune disease.

The bispecific molecule of the present invention may be used alone in the treatment of arthritis. The bispecific molecule may have intrinsic anti-angiogenic activity, for example it may be capable blocking essential mediators of vascular proliferation. Examples of such agents currently in clinical trials are drugs capable of neutralizing anti-VEGF antibodies and antibodies directed against a VEGF receptor or the αvβ3 integrin.

Alternatively the bispecific molecule may be used in a combination therapy with another agent (see below).

### Combination Therapies

The bispecific molecule of the present invention may be used in combination with another therapy. The two therapeutic agents may be for separate, subsequent or simultaneous administration.

The other therapy may comprise a therapeutic cytokine, an anti-angiogenic agent or an anti-rheumatic drug, as described above.

The bispecific molecule of the present invention may be used in combination with another recombinant antibody used for the treatment of arthritis.

Currently, there are several recombinant antibodies in use for treatment of Rheumatoid Arthritis, targeting a range of cytokines, T cells and B cells. Since the initial approval of Etanercept, and shortly thereafter Infliximab, three additional TNF-neutralizing antibodies (Adalimumab, Certulizumab pegol and Golimumab) have been approved. Further, recombinant antibodies targeting T-cell [and/or dendritic cell], (Abatacept), B-cells, (Rituximab), and the receptor for cytokine IL-6, (Tocilizumab) have also been approved by the FDA for treatment of RA (Taylor and Feldmann 2009; Isaacs 2009 both as above).

The other treatment may involve targeting T cells, dendritic cells, B-cells and/or IL-6 using the antibodies described above. Alternative antibodies providing the same function may also be used.

### Kits

Also described is a kit comprising a bispecific molecule in accordance with the first aspect of the invention.

Where the bispecific molecule is for diagnostic use, the kit may also comprise further imaging reagents and/or apparatus.

Where the kit is for use in a combination therapy, the kit may also comprise a second therapeutic agent for simultaneous, subsequent or separate administration.

### Imaging

The bispecific molecule may be used in imaging applications, for example in imaging the vasculature of arthritic joints.

To date, only few good-quality markers of angiogenesis, either on endothelial cells or in the modified ECM, are known. The biggest problem with many of the markers is that they lack sufficient specific expression or significant upregulation in tissues undergoing angiogenesis.

Some integrins, in particular αvβ3 and αvβ5, have been proposed both as markers and as functional mediators of angiogenesis in tumors and in ocular neovascular disorders. Expression of integrin αvβ3 was also shown to be increased in synovial blood vessels from patients with rheumatoid arthritis. However, in recent immunohistochemical studies, the vasculature in apparently normal tissue as well as several extravascular cell types were shown to stain positive for αvβ3, even though at lower intensity than in tissues undergoing angiogenesis.

Many recent studies have described endoglin (CD105), a component of the transforming growth factor-β receptor complex, as an attractive marker of neovascularization. Endoglin shows considerably increased expression on proliferating endothelium, but it also weakly stains endothelial cells in the majority of normal, healthy adult tissues of both human and mouse origin. Several monoclonal antibodies to endoglin have been characterized and have recently been tested as targeting agents for therapy and imaging of tumors. Unexpectedly, the targeting results obtained in mice were relatively modest, in spite of the accessible localization of the antigen on endothelial cells.

There is thus a need for improved agents for imaging the microvasculature of arthritic joints.

The bispecific molecule of the invention may be labelled for imaging techniques, with, for example a fluorescent or radioactive label.

In vivo imaging techniques using antibodies are well known in the art, including bioluminescence imaging (BLI) and biofluorescence imaging (BFI).

### Diagnostic Methods

The bispecific molecule may be used in a method for diagnosing a disease.

The bispecific molecule may be used in a method for monitoring the progression of a disease and a method for evaluating the efficacy of a drug treatment.

The disease may be associated with a change, for example an increase, in the synovial microvasculature. The disease may be a form of arthritis, such as osteoarthritis or rheumatoid arthritis.

As explained in the background section, synovial angiogenesis is likely to precede other pathological features of RA, so the bispecific molecule of the present invention may be useful for the diagnosis of RA at an early stage, prior to the appearance of other symptoms.

The method may involve imaging the synovial microvasculature of a joint of the patient at one or a plurality of time points.

The invention will now be further described by way of Examples, which are meant to serve to assist one of ordinary skill in the art in carrying out the invention and are not intended in any way to limit the scope of the invention.

### EXAMPLES

### Example 1

### Generation of scFv-A7-Fc and its Coupling with Adalimumab scFv to Produce a Bispecific Antibody

The present inventors have developed a bispecific antibody for A7/Adalimumab using Knobs-into-Holes technology.

The sequence for scFvA7 (originally derived from phage display using the Tomlinson library and produced by E. coli) was optimised for Chinese Hamster Ovary (CHO) expression, using GeneArt DNA synthesis service (Life Technologies). The sequences for the V_{H} and V_{L} domains of Adalimumab were obtained from WO 97/29131. The scFv format sequence was optimised for CHO expression and synthesised using GeneArt service, linking the two variable domains with a serine-glycine linker (SSGGGGSGGGGSGGGGS) in V_{H}-V_{L} orientation.

The scFvA7 antibody fragment was fused with the hinge, C_{H}2 and C_{H}3 domains of Human IgG1 carrying the T366Y mutation (Knob). The Adalimumab derived scFv sequence was fused with the hinge, C_{H}2 and C_{H}3 domains of Human IgG1 carrying the Y407T mutation (Hole).

scFv-Fc fusion protein sequences for both A7 and Adalimumab were inserted into pCDNA3.1Hygro(+)(Invitrogen) to form a single monocystronic gene. To this end, an IgG secretory leader sequence of 20aa was inserted before the Adalimumab scFv-Fc, a mini intron was introduced into the DNA sequence between the leader sequence and the scFv to increase transcription efficiency, and a SV5 tag was inserted at the end. The A7 scFv-Fc portion was fused to the Adalimumab scFv-Fc via the 2A peptide sequence (24aa sequence APVKQTLNFDLLKLAGDVESNPGP derived from Food and Mouth Disease Virus) and a second IgG secretory leader with a mini intron was inserted between the 2A peptide and the A7 scFv-Fc sequence. This second scFv-Fc sequence also comprises a 6 Histidine tag.

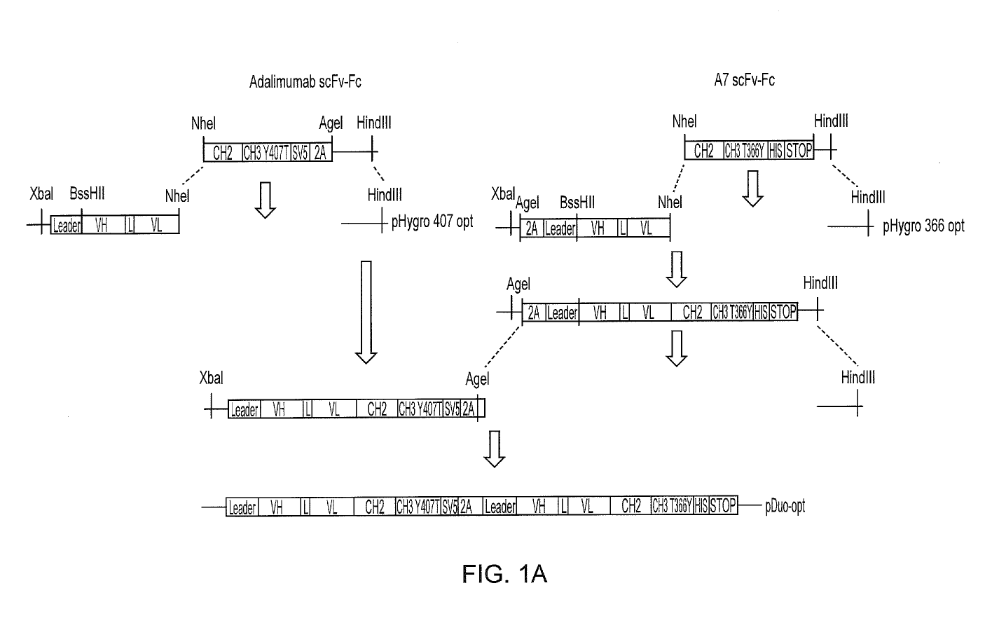

A schematic for the cloning strategy adopted is provided in FIG. 1.

A single mRNA is obtained upon transcription of the bispecific gene. The first leader peptide provides the signal for secretion of the first scFv-Fc molecule, whilst the 2A sequence allows the ribosome to skip one codon and thus release the first peptide chain before continuing with the second scFv-Fc sequence where the second leader peptide provides the signal for secretion. Residual amino acid residues from the 2A peptide are cleaved by the Furin protease. This strategy allows a 1:1 ratio for the two scFv-Fc molecules, increasing the efficiency of heterodimerisation.

The vector containing the bispecific antibody construct was used to transfect a CHO-s cell line and a stably transfected cell line was obtained through the use of Hygromycin B as a selective agent. The bispecific antibody was then purified from the transfected CHO cell line culture supernatant using TALON metal affinity chromatography (Clonetech).

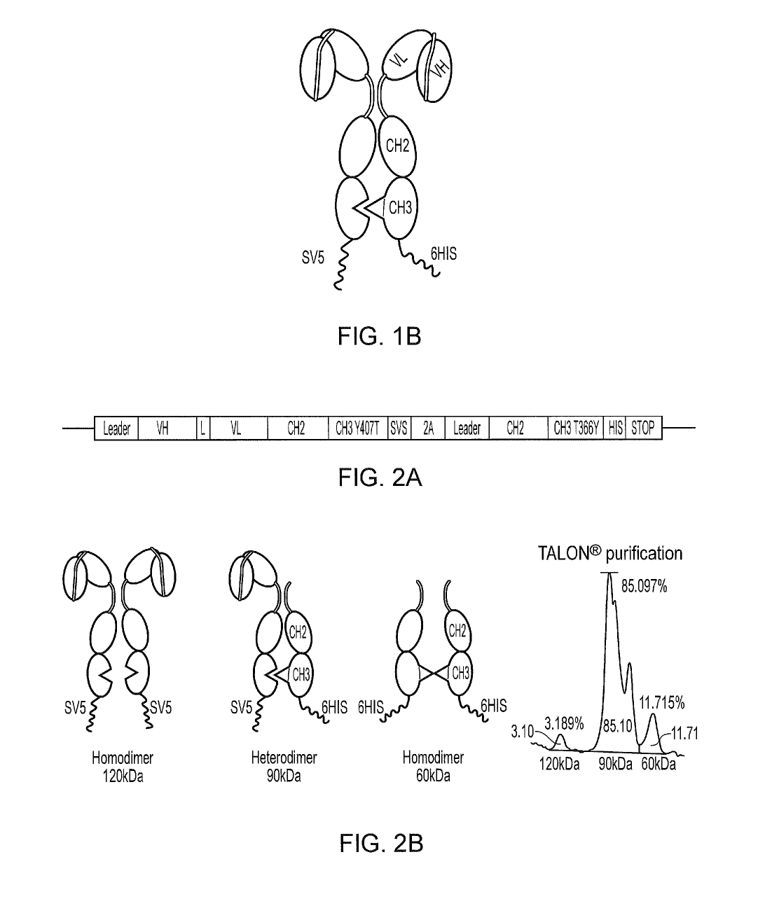

The heterodimerisation efficiency that can be obtained using the Knobs-into-Holes technology depends on the ratio between the two chains and on the antibody to be produced. To calculate the dimerization obtained with the present construct, the scFvA7 was deleted from the peptide sequence in order to form an asymmetric bispecific antibody. This construct enabled the identification of the 3 possible dimers (heterodimer and homodimer for either of the two chains, FIG. 2A). Analysis of the antibody purified in a non-reducing SDS-PAGE demonstrated a high degree of efficient heterodimerisation (85% heterodimers, FIG. 2B).

### Example 2

### The Reactivity of the A7/Adalimumab Bispecific Antibody on Tissue Sections

Bispecific antibody reactivity on tissue was assessed in paraffin embedded formalin fixed tissue section and in OCT embedded frozen sections using immunohistochemistry (IHC).

Paraffin embedded tissue sections of human arthritic synovium were used for the testing of bispecific A7/Adalimuab antibody reactivity in comparison to A7 scFv-Fc and Adalimumab scFv-Fc antibodies independently. Tissue sections were dewaxed and the antigen was retrieved using proteinase K enzymatic reaction. Endogenous peroxidase activity was blocked using 3% H₂O₂ in methanol and non-specific protein binding sites were blocked using a protein block solution. Bound biotinylated antibodies on the tissue were detected using streptavidin-HRP.

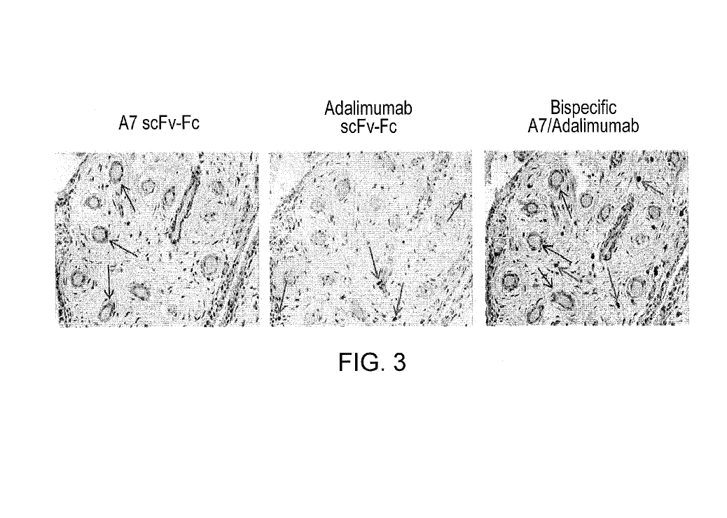

A representative staining in arthritic synovium is shown in FIG. 3. A7 reactivity was confined in the vascular region of the synovium (blue arrows), while the Adalimumab reactivity was specific for a TNF producing cell subset (red arrows). Compared to A7 scFv-Fc and Adalimumab scFv-Fc, the bispecific antibody A7/Adalimumab show both specificities in the synovium, demonstrating the functional activity of the two chains.

OCT embedded frozen tissue sections of human arthritic synovium were also used for the testing of bispecific A7/Adalimuab antibody reactivity in comparison to A7 scFv-Fc and Adalimumab scFv-Fc antibodies independently.

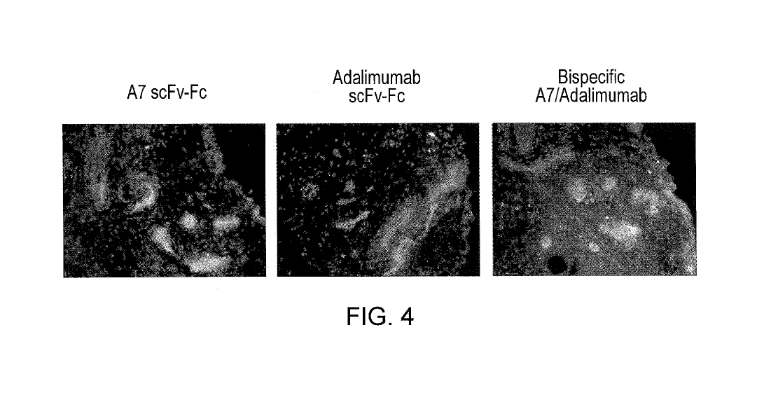

The sections were fixed in ice cold acetone and blocked for non specific protein binding sites using a protein block solution. Bound biotinylated antibodies were detected using streptavidin-ALEXA fluor 488. Antibody against the human vWF was detected using anti-mouse ALEXA fluor 555 conjugated antibody. FIG. 4 shows a representative dual staining in arthritic synovium in the presence of anti-vWF.

A7 reactivity was confined in the vascular region of the synovium (green). The bispecific antibody A7/Adalimumab showed a similar reactivity on the synovium to A7 scFv-Fc, demonstrating the functional activity of the A7 portion.

### Example 3

### In Vivo Dosage and Administration Efficiency of the Bispecific Antibody

The in vivo localisation of the scFv-Fc bispecific antibody to the tissue of interest is demonstrated using time-domain near-infrared optical imaging.

This demonstrates that the bispecific molecule preferentially targets the inflamed synovium over anti-TNF monovalent antibody. Localisation data is be coupled with pharmacokinetic data showing that antibody clearance is not affected by the manipulation of the antibody to form a bispecific compound. Pharmacokinetic measurements is used to demonstrate the antibody clearance rate in mice.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in autoimmunity, antibody technology, molecular biology or related fields are intended to be within the scope of the following claims.

1. A bispecific molecule comprising:
(i) a first antigen binding portion which specifically targets the synovial microvasculature of arthritis patients and which binds to the same epitope as an antigen binding polypeptide comprising the amino acid sequence shown as SEQ ID No 11; and
(ii) a second antigen binding portion which binds tumour necrosis factor alpha (TNF-α).

2. A bispecific molecule according to claim 1 wherein the first antigen binding portion comprises:
a) a heavy chain variable region comprising
(i) a CDR1 comprising SEQ ID No. 1;
(ii) a CDR2 comprising SEQ ID No 2; and
(iii) a CDR3 comprising SEQ ID No 3, and
b) a light chain variable region comprising
(i) a CDR1 comprising SEQ ID No. 4;
(ii) a CDR2 comprising SEQ ID No. 5; and
(iii) a CDR3 comprising SEQ ID No. 6
or a variant of any one or more of those CDR sequences having up to three amino acid variations from the given sequence, provided that the first antigen binding portion retains the ability to bind to the same epitope as an antigen binding polypeptide comprising the amino acid sequence shown as SEQ ID No 11.

3. A bispecific molecule according to claim 1 or 2 wherein the first antigen binding portion comprises a VH sequence as shown in SEQ ID No. 9 and a VL sequence as shown in SEQ ID No. 10, or a variant thereof having at least 80% sequence identity which specifically targets the synovial microvasculature of arthritis patients and which binds to the same epitope as an antigen binding polypeptide comprising the amino acid sequence shown as SEQ ID No 11.

4. A bispecific molecule according to any preceding claim wherein the second antigen binding portion comprises:
a) a heavy chain variable region comprising
(i) a CDR1 comprising SEQ ID No. 7;
(ii) a CDR2 comprising SEQ ID No 8; and
(iii) a CDR3 comprising SEQ ID No 12, and
b) a light chain variable region comprising
(i) a CDR1 comprising SEQ ID No. 13;
(ii) a CDR2 comprising SEQ ID No. 14; and
(iii) a CDR3 comprising SEQ ID No. 15
or a variant of any one or more of those CDR sequences having up to three amino acid variations from the given sequence, provided that the second antigen binding portion retains the ability to bind TNF-α.

5. A bispecific molecule according to any preceding claim wherein the second antigen binding portion comprises a VL sequence as shown in SEQ ID No. 16 and a VH sequence as shown in SEQ ID No. 17 or a variant thereof having at least 80% sequence identity which is capable of binding TNFα.

6. A bispecific molecule according to any preceding claim which is an scFv.

7. A bispecific molecule according to any preceding claim which is a bispecific human antibody.

8. A bispecific molecule according to any preceding claim for use in the treatment of arthritis.

9. A method for treating arthritis in a subject, which comprises the step of administering a bispecific molecule according to any of claims 1 to 7 to a subject.

10. A method according to claim 9 for treating osteoarthritis and/or rheumatoid arthritis.

11. A method for producing a bispecific molecule according to any of claims 1 to 7, which method comprises the step of conjugating the first antigen binding portion to the second antigen binding portion.

12. A nucleic acid sequence encoding a bispecific molecule according to any of claims 1 to 7.

13. A vector comprising a nucleic acid sequence according to claim 12.

14. A host cell comprising a vector according to claim 13.
